(19) United States
(12) Patent Application Publication
(10) Pub. No.: US 2023/0071643 A1
(43) Pub. Date: Mar. 9, 2023

(54) Ultrasound Diaphragmography Device and Method

(71) Applicant: Daniel Nathan Maxwell, Dallas, TX (US)

(72) Inventor: Daniel Nathan Maxwell, Dallas, TX (US)

(21) Appl. No.: 17/842,784

(22) Filed: Jun. 17, 2022

Related U.S. Application Data
(60) Provisional application No. 63/223,959, filed on Jul. 20, 2021.

(51) Int. Cl. A61B 8/08; A61B 8/00; B06B 1/06

(52) U.S. Cl. CPC A61B 8/085; A61B 8/4477; A61B 8/54; A61B 8/5207; A61B 8/461; A61B 8/4236; A61B 8/4494; A61B 8/4488; B06B 1/0622; B06B 2201/76

(57) ABSTRACT

An ultrasound diaphragmography device includes a plurality of piezoelectric ultrasound transducers arranged in at least two linear arrays for placement on a patient's chest along a cranio-caudal axis of the patient. The ultrasound transducers generate and transmit ultrasound waves that penetrate the patient's anatomy and receive returned ultrasound waves. Circuitry in communication with the ultrasound transducers receive the returned ultrasound waves and analyzes the returned ultrasound waves to determine the quality of the patient's lung function in real-time and compare the movement of the left and right hemidiaphragms.

### RELATED PATENT APPLICATION

This patent application claims the benefit of U.S. Provisional Patent Application No. 63/223,959 filed on Jul. 20, 2021, the entirety of which is incorporated herein by reference.

### FIELD

The present disclosure primarily relates to the field of medical devices, and more particularly to an ultrasound diaphragmography device and method with a variety of use cases.

### BACKGROUND

Despite its physiologic importance, measurement of respiratory rate and lung function in non-ventilated patients lags in precision and accuracy behind other vital signs. Heart rate, temperature, weight, and blood pressure can all be measured through multiple advanced modalities, while respiratory rate and lung function in non-ventilated patients is still monitored in practice almost exclusively through visual observation of chest movement by the clinician. The continued use of manual observation, unchanged for over a century, is indicative of the flaws and disadvantages of existing technologies such as belt plethysmography, respiratory inductive or optoelectronic plethysmography, and wearable strain sensors, which all have limitations that have prevented widespread clinical adoption.

### DESCRIPTION OF THE DRAWINGS

FIG. 1 is schematic block diagram of an exemplary embodiment of the ultrasound diaphragmography device according to the teachings of the present disclosure;

FIG. 2 is another schematic block diagram of an exemplary embodiment of the ultrasound diaphragmography device according to the teachings of the present disclosure; and

FIG. 3 is an illustration of an exemplary embodiment of the ultrasound diaphragmography device placed on the patient's chest during operations of the device according to the teachings of the present disclosure.

### DETAILED DESCRIPTION

Ultrasound has been deployed in medical devices to obtain high-resolution 2D and 3D images of patients' internal anatomy. However, ultrasound is not generally employed as a tool to measure respiratory rate, diaphragmatic movement, and lung function in a clinical setting, despite some known techniques. Ultrasound devices that use a single array of ultrasound transducers have been previously described as a method to monitor the respiratory rate (e.g., U.S. Pat. No. 8,814,793). However, such devices have two primary limitations. First, though the acoustic impedance of the lung and the abdomen diverge by several orders of magnitude, the rib cage causes significant acoustic shadowing. This, along with other sources of noise such as bowel gas and transducer positioning in obese patients, reduces the resolution of such an approach considerably, making measurement with a single transducer array challenging. Second, the single-array devices do not allow for comparison of the movement of the left and the right hemidiaphragms. Using at least two ultrasound transducer arrays placed on the left and the right side would allow for a comparison of the movement of the left and the right hemidiaphragms, which also has the advantage of requiring much lower resolution than measurement of the respiratory rate using a single transducer array. Specifically, with the application of bilateral ultrasound transducer arrays, one could ensure gross movement of both the left and the right hemidiaphragms, rather than attempting to measure their precise rate of movement with this safe, low cost, but low-resolution technology. This approach would allow accurate detection of hemidiaphragmatic paralysis or weakness as well as endobronchial intubation.

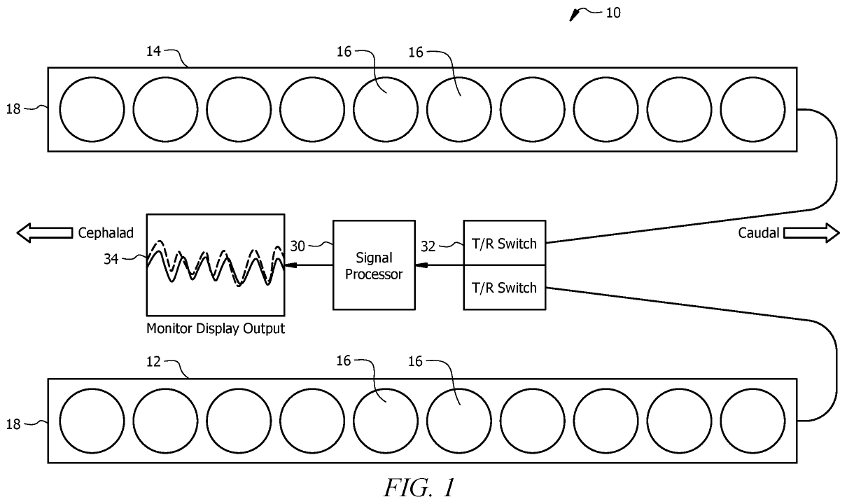

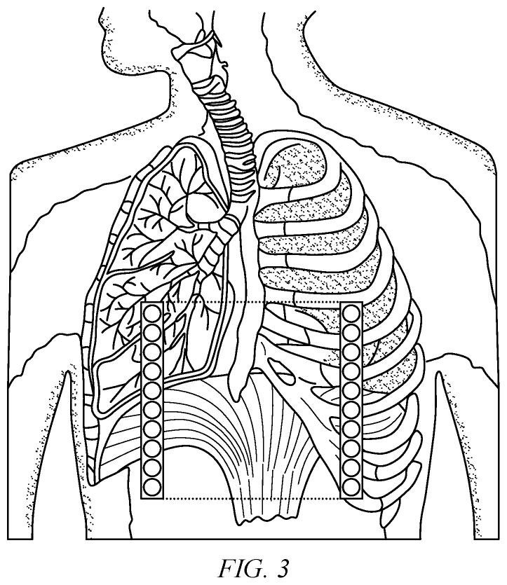

Referring to FIG. 1, the ultrasound diaphragmography device 10 includes two or more linear arrays 12 and 14 of small (˜10 mm) piezoelectric ultrasound transducers 16 (aka transceivers) (e.g., 3 MHz) contained within a protective sleeve or housing (hereinafter generally referred to as sleeve) 18, to be placed or adhered to the patient's chest along the cranio-caudal axis. Each transducer array 12 and 14 may include two or more transducers 16 that may or may not be spaced equidistantly. The sleeve may be flexible or rigid, and the sleeves housing the two sensor arrays may be separate components or connected. For example, the two arrays may be connected or linked in a generally parallel manner, such as shown in FIG. 3.

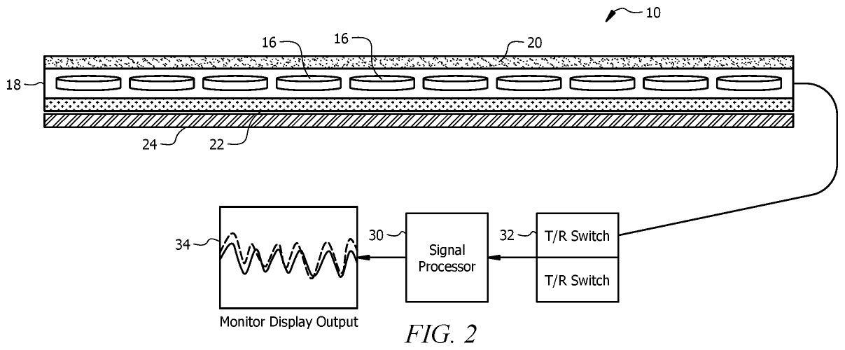

Referring to FIG. 2, the ultrasound transducers 16 are preferably arranged in each sleeve 18 with a spacing that is suitable for the transmission, conduction, and reception of ultrasound waves with minimal interference from neighboring transducers. The sleeve 18 may include an ultrasound backing material 20 on the side intended to face away from the patient's chest (backside) and an ultrasound coupling or conductive material 22 on the side intended to face toward the patient's chest (front side). The sleeves 18 are preferably constructed of suitable materials that help to direct ultrasound waves generated by the transducers 16 toward the patient-facing side of the sleeve, absorb ultrasound waves to minimize errant ultrasound echoes, and/or to reduce ultrasound wave interference between transducers. The material on the patient-facing side of the sleeve 18 is preferably conductive of ultrasound wave energy. On the exterior of the sleeve 18 on the patient-facing side may further incorporate an ultrasound coupling/conductive material 24 that has adhesive properties to adhere the sensor arrays 12 and 14 to the patient's chest.

As shown in FIG. 3, the ultrasound diaphragmography device 10 may include two transducer arrays 12 and 14 that are placed over each of the patient's lungs to individually monitor and measure their ability to move air in and out. The transducer arrays 12 and 14 are preferably placed on either side of the centerline of the patient's chest over the rib cage, preferably spanning or covering all possible positions where the patient's diaphragm may be located. The length spanned by each transducer array may be, for example, 10-14 cm. The transducer arrays 12 and 14 are thus arranged along the cranio-caudal axis of the patient's diaphragm movement so that as the patient breathes in and out, the position and movement of the diaphragm can be dynamically detected by the transducer arrays 12 and 14.

The transducers 16 of the ultrasound diaphragmography device 10 are configured to generate and transmit ultrasound signals of a certain frequency that are directed toward the patient's body anatomy. The ultrasound waves penetrate the patient's body and impinge on the patient's ribs, lungs, diaphragm, liver and other tissues, and are reflected back as acoustic echoes. Because the speed, direction, and distance sound waves travel differ depending on the tissue boundaries they run into, the returned sound waves are received by the transducers as measurements of acoustic impedance in Rayls (Z) or MegaRayls (MZ) and transmitted to a signal processor 30 (e.g., CPU or a specialized signal processor) coupled to the transducer arrays 12 and 14. Transmit/receive switches (T/R switches) 32 may be used to control the transmit and receive functions of the transducers 16. The signal processor 30 interprets the received signal to distinguish what types of tissues were detected. These acoustic impedance measurements are dynamically (i.e., in real-time) categorized by the signal processor 30 into one of three categories: air, tissue, or bone. Because the acoustic impedances of these elements are widely divergent, with air at 0.0004 MZ, lung tissues at approximately 0.18 MZ, liver tissues at 1.65 Z, adipose at 1.34 Z, and bone at 7.8 Z, a wide margin (three orders of magnitude) separates the acoustic impedance for tissue and air and a wide margin (one order of magnitude) still separate lung from other, non-aerated tissues. This allows for a relatively easy task of tissue classification. If a transducer in a sensor array detects bone, the transducer is assumed to be overlying a rib and its measurements will be excluded. When the device is measuring air or tissue and that signal changes categories (between air to tissue or vice versa) this is indicative the movement of the diaphragm past the field of detection of that transducer. By analyzing such changes among each array of transducers, the movement of the diaphragm can be deduced with clinically meaningful accuracy in real-time using low-cost components. The degree of diaphragmatic movement on the left and the right hemidiaphragm can then be compared, even in cases of degraded signal quality.

The signal processor 30 may generate an output that is displayed on a screen 34. The output may include still images, moving images, text, numerical data, and audible sound that are indicative of the comparative movement of the left and the right hemidiaphragm. The output may further include audible alarms or alert text messages when the patient's diaphragmatic movement is asymmetric and requires urgent medical attention. Other components of the ultrasound diaphragmography device 10 may include, for example, ultrasound pulse control, analog circuitry, A/D converter, memory, wired or wireless communications interfaces between components (e.g., between the ultrasound transducers and T/R switches and/or between the T/R switches and the signal processor), user interface devices (e.g., keyboard, touch display screen, mouse, pointer, speaker, printer), and disk storage devices.

The proposed ultrasound diaphragmography device 10 offers the benefits of accurate and continuous measurement of the movement of the left and the right hemidiaphragm. This technology has several clinical applications.

Endotracheal tubes are used to intubate patients and connect them to mechanical ventilation. Though there are several technologies to confirm placement of the endotracheal tube within the trachea, such as capnography and colorimetric CO2 detection, there are currently no technological adjuncts in clinical use to assess for endobronchial or “main stem” intubation aside from chest X-ray. Endobronchial intubation is a condition in which the endotracheal tube has been advanced beyond the carina and ventilation is subsequently delivered to only one of the lungs. This event has been described as occurring as frequently as 15% of cases (PMID 33868864) and can cause significant morbidity and even mortality. Though there are clinical techniques to assess for endobronchial intubation, such as auscultation of both lungs, these techniques can be affected by ambient noise, whether in an emergent hospital setting or in the field. The technology described here could detect endobronchial intubation safely and reliably by detecting asymmetric or unilaterally absent hemidiaphragmatic movement.

Another application of this novel device is assessing for hemidiaphragmatic paralysis. Weakness of one diaphragm can be seen after trauma, especially surgical trauma. Lung cancer can also be complicated by weakness of one hemidiaphragm. This device would be able to compare the movement of the two hemidiaphragms. This ultrasound diaphragmography device 10 may also be used to provide additional information. For example, the quality of air intake of the lungs, the pattern of respiration, etc. may also be measured and provided as output. Telehealth, sleep study (e.g., for sleep apnea), and other applications are also foreseeable. This device does not require ionizing radiation, can be placed on the right anterior chest, the left anterior chest, or both, and can be delivered at significantly lower cost due to the lower demands in resolution and signal-to-noise ratio.

Yet another application for this device is for monitoring fluid (i.e., liquids) management in patients with chronic heart failure. Pulmonary congestion, or fluid overload, is a classic clinical feature of patients presenting with heart failure exacerbation, and its presence is associated with adverse outcomes. However, as congestion is not always clinically evident, more objective measures of congestion than simple clinical examination would be helpful. Fluid in the base of the patient's lungs may be detected by using the proposed diaphragmography device to measure an increase in the acoustic impedance of the lung tissue where fluid has accumulated. If the ultrasound diaphragmography device detects a change in the acoustic impedance indicative of a change in the detected characteristic of the underlying tissues when the patient changes between a supine position and head-of-bed elevated position, it may be indicative of a shift in the pooled fluids in the patient's lungs. Further, as the patient is being treated with diuretic medication and fluid is removed, the acoustic impedance of the lung tissue will also decrease toward baseline.

It should be noted that the transducers may be arranged in an array as shown, or they may be arranged in other suitable formations, such as a matrix.

The features of the present invention which are believed to be novel are set forth below with particularity in the appended claims. However, modifications, variations, and changes to the exemplary embodiments of the ultrasound diaphragmography device and method described above will be apparent to those skilled in the art, and the protection device described herein thus encompasses such modifications, variations, and changes and are not limited to the specific embodiments described herein.

What is claimed is:

1. An ultrasound diaphragmography device, comprising:
a plurality of piezoelectric ultrasound transducers arranged in a predetermined fixed pattern for placement on a patient's chest along a cranio-caudal axis of the patient;
a housing containing the plurality of piezoelectric ultrasound transducers and having an ultrasound backing layer disposed along a back inside surface of the housing and an ultrasound coupling material disposed along a front inside surface of the housing;
a transmit/receive switch coupled to the plurality of piezoelectric ultrasound transducers configured to control the transmit and receive operations of the plurality of piezoelectric ultrasound transducers to transmit ultrasound waves directed outward from the front of the housing toward the patient's chest and receive ultrasound waves returned from the patient's chest; and
a signal processor coupled to the transmit/receive switch configured to receive the returned ultrasound waves and analyze the returned ultrasound waves to dynamically determine the quality of the patient's lung function.

2. The device of claim 1, wherein the plurality of piezoelectric ultrasound transducers are arranged in at least two linear arrays.

3. The device of claim 1, wherein the signal processor is configured to dynamically determine a location of an interface between air in the patient's lungs and adjacent tissues.

4. The device of claim 1, further comprising an output device coupled to the signal processor configured to present information indicative of the quality of the patient's lung function.

5. The device of claim 1, wherein the housing further comprises an ultrasound coupling adhesive configured for adhering the housing to the patient's chest.

6. The device of claim 1, wherein the plurality of piezoelectric ultrasound transducers are arranged in at least two separate linear arrays where each linear array is enclosed in a dedicated housing, and the two linear arrays and housing are configured for placement along a cranio-caudal axis of the patient over each of the patient's lungs.

7. The device of claim 1, wherein the signal processor is configured to dynamically detect and identify a location of the patient's diaphragm and generate an output indicative of information selected from the group consisting of the patient's respiratory rate and the lung's location.

8. The device of claim 7, further comprising an output device coupled to the signal processor configured to present information indicative of the quality of the patient's lung function selected from the group consisting of still images, moving images, text, numerical data, and audible sound.

9. A method for ultrasound diaphragmography, comprising:
transmitting a plurality of ultrasound sound waves from a plurality of ultrasound transducers arranged in at least one linear array for placement on patient's chest along a cranio-caudal axis of the patient and directing the plurality of ultrasound sound waves toward the patient's chest;
receiving ultrasound waves returned from the patient's chest; and
analyzing the returned ultrasound waves to dynamically determine the quality of the patient's lung function.

10. The method of claim 9, further comprising presenting information selected from the group consisting of still images, moving images, text, numerical data, and audible sound indicative of the quality of the patient's lung function.

11. The method of claim 9, wherein analyzing the returned ultrasound waves comprises determining whether a particular transducer at a particular location in each of the at least one linear array is detecting one of lung tissue, liver tissue, bone, adipose, liquid, and air.

12. The method of claim 9, wherein analyzing the returned ultrasound waves comprises determining a location of an interface between air in the patient's lungs and adjacent tissues.

13. The method of claim 9, wherein analyzing the returned ultrasound waves comprises determining a dynamic location of the patient's diaphragm.

14. The method of claim 9, wherein analyzing the returned ultrasound waves comprises determining presence of liquids in the patient's lungs.

15. The method of claim 9, further comprising minimizing or accounting for ultrasound wave interference between transducers.

16. An ultrasound device for dynamically monitoring proper functioning of a patient's lungs, comprising:
a plurality of ultrasound transducers arranged in at least one linear array for placement on a patient's chest over the patient's diaphragm along a cranio-caudal axis;
a controller coupled to the plurality of piezoelectric ultrasound transducers configured to control the transmit and receive operations of the plurality of ultrasound transducers to transmit ultrasound waves directed toward the patient's chest;
a signal processor in electrical communication with the controller configured to receive the returned ultrasound waves and analyze the returned ultrasound waves to dynamically determine the quality of the patient's lung function; and
an output device in electrical communication with the signal processor configured to present data indicative of the quality of the patient's lung function.

17. The device of claim 16, wherein the plurality of ultrasound transducers are arranged in at least two separate linear arrays where each linear array is enclosed in a dedicated housing, and the two linear arrays and housing are configured for placement along a cranio-caudal axis of the patient over each of the patient's lungs.

18. The device of claim 16, wherein the signal processor is configured to dynamically detect and determine a location of the patient's diaphragm and generate an output indicative of information selected from the group consisting of the patient's respiratory rate and the lung's location in real-time.

19. The device of claim 18, wherein the output device is configured to present information indicative of the quality of the patient's lung function selected from the group consisting of still images, moving images, text, numerical data, and audible sound.

20. The device of claim 16, further comprising a flexible sleeve for housing the at least one linear array of ultrasound transducers and the sleeve further comprises an ultrasound coupling adhesive for adhering to the patient's chest.
